(19) United States
(12) Patent Application Publication
(10) Pub. No.: US 2018/0153439 A1
(43) Pub. Date: Jun. 7, 2018

(54) Time-Resolved Single-breath Analysis Using Spectroscopic Methods

(71) Applicant: The Board of Trustees of the Leland Stanford Junior University, Palo Alto, CA (US)

(72) Inventors: Victor A. Miller, Los Angeles, CA (US); Raymond M. Spearrin, Calabasas, CA (US); Christopher L. Strand, San Mateo, CA (US); Gregory M. Enns, Redwood City, CA (US)

(21) Appl. No.: 15/577,709

(22) PCT Filed: Jun. 8, 2016
(86) PCT No.: PCT/US16/36457

§ 371 Date: Nov. 28, 2017

Related U.S. Application Data
(60) Provisional application No. 62/172,710, filed on Jun. 8, 2015.

(51) Int. Cl. A61B 5/083; A61B 5/08; A61B 5/00; A61B 5/097; G01N 21/31; G01N 21/85; G01N 33/497; G01N 1/22

(52) U.S. Cl. CPC A61B 5/083; A61B 5/082; A61B 5/0075; A61B 5/097; G01N 21/31; G01N 2001/2244; G01N 33/4972; G01N 1/22; G01N 2021/8578; G01N 2033/4975; G01N 21/85

(57) ABSTRACT

Time-resolved continuous detection of gas species concentration in breath is provided by optical spectroscopy. Preferably this is performed for both target and reference chemical species, so that the reference measurements can be used to assist in identifying breath cycle phases and to correct the target measurements for contamination by environmental air. Identification of breath cycle phases can be used to gate the data to select the most relevant parts (e.g., the data from when the contribution from alveolar air is maximal).

### FIELD OF THE INVENTION

This invention relates to breath analysis.

### BACKGROUND

Breath analysis is of general interest for a wide variety of medical analyses. For some exhaled breath analytes, such as ammonia, knowledge of the phase or depth of exhalation is important for alveolar concentration analysis. Exhaled carbon dioxide (Ex-CO₂) is an accepted reference parameter for the determination of (1) dilution of a breath sample and (2) phase of the exhaled breath. In the latter case, time-resolved detection of gas-phase Ex-CO₂ is required (˜0.1 second time resolution). An independent time-resolved measure of Ex-CO2 is commonly collected with a capnograph, and used for partitioning the breath for subsequent analysis (commonly ‘end-tidal’ analysis) or for normalization to correct for dilution. The effectiveness of either of these breath sampling control methods (phase analysis and dilution correction) depends on proper time synchronization between the various components (valves, etc.) or sensors (time-lag correction).

It would be an advance in the art to provide improved time-resolved breath sampling, especially of multiple species in a single breath.

### SUMMARY

Here we present a simplified approach to measure multiple species simultaneously and with synchronous time resolution using laser absorption spectroscopy, enabling accurate and real-time analysis of phase and dilution via intra-breath resolved gas detection. This method reduces complexity of a multi-sensor system, and improves time-synchronization between the species. The approach is particularly well-suited to rapid-response, point-of-care analysis and in instances where short breaths need to be resolved (e.g., for children).

One application for this technology is ammonia monitoring for managing inborn errors of metabolism (IEM). Inborn errors of metabolism, such as a urea cycle disorder or organic acidemia, are congenital disorders that predispose those who have one to certain health issues, like hyperammonemia, which can acutely manifest in an emergency situation. Hyperammonemia (elevated levels of ammonia in the blood stream) resulting from an IEM, if misdiagnosed or diagnosed late can lead to seizure, permanent neurological damage, coma, and death, and so rapid diagnosis and monitoring of hyperammonemia is crucial for patients with IEMs. This work provides patients, physicians, and caretakers a way to manage complications resulting from IEMs using regular, non-invasive concentration measurements of specific compounds in the breath, compounds which correlate to the state of the complication.

Other medical conditions related to kidney and liver function (e.g., dialysis, cirrhosis, diabetic ketoacidosis) or the metabolism could potentially also be managed or diagnosed using a breath-measurement of relevant species. Management of complications could entail determination of treatment efficacy, optimization of treatment, and impetus to administer preventative treatment.

Existing methods for management of IEM complications include behavioral observation and blood assays. Behavioral observation is non-specific (e.g., mild hyperammonemia can manifest in lethargy or hyperactivity), and blood assays are painful, inconvenient, and notoriously inaccurate. A breath-based measurement is non-invasive and has negligible variable cost, and a breath sample can be easily provided by children (IEMs commonly affect children) or patients with neurological injury.

### BRIEF DESCRIPTION OF THE DRAWINGS

FIG. 1 is a schematic block diagram of embodiments of the invention.

FIG. 2 shows an exemplary embodiment of the invention for oral breath sampling.

FIG. 3 shows an exemplary embodiment of the invention for nasal breath sampling.

FIG. 4 shows illustrative time-resolved results from oral breath sampling.

FIG. 5 shows illustrative time-resolved results from nasal breath sampling.

### DETAILED DESCRIPTION

An exhaled breath analysis technology has been developed to enable the time-resolved measurement of the intra-breath composition over an entire exhalation. This measurement capability permits the identification and analysis of systemically relevant breath phases, the correction for sample contamination, the incorporation of biofeedback to improve sample quality, and the rejection of poor quality samples.

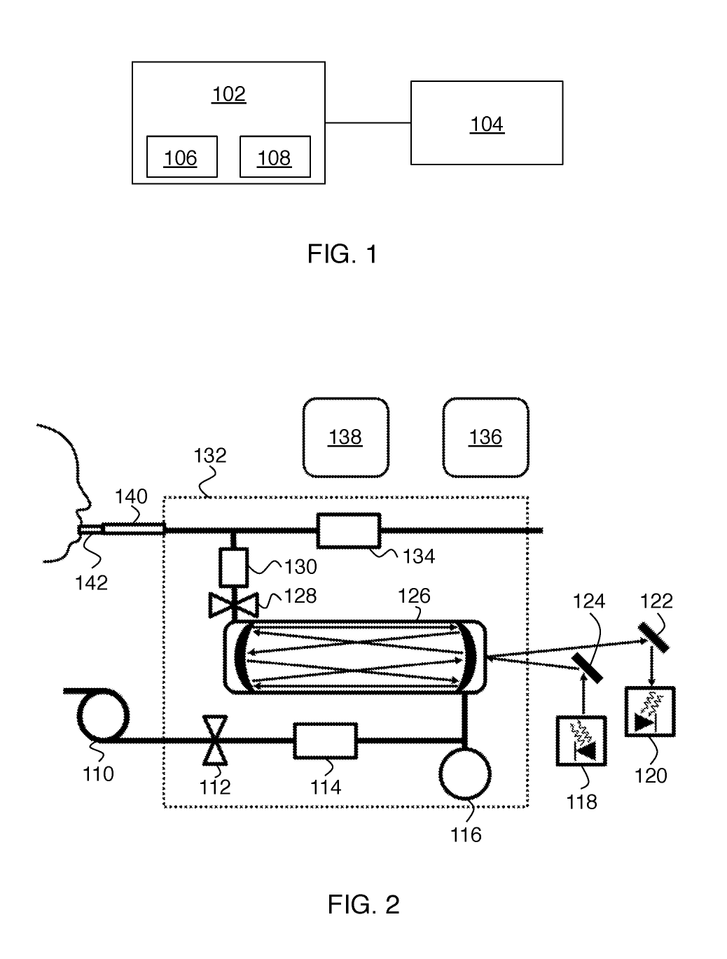

FIG. 1 shows a schematic block diagram. Here 102 is a breath sample collection unit, which preferably includes flow rate sensor 106 and/or pressure sensor 108. 104 is an optical spectrometer configured to receive gas from the breath sample collection unit 102 and to provide time-resolved and continuous intra-breath concentration measurements of at least one target chemical species. The optical spectrometer is preferably further configured to provide time-resolved and continuous intra-breath concentration measurements of at least one reference chemical species, where the measurements of the target species and the reference species are synchronized.

The present approach is applicable to any spectroscopically active molecule and is particularly important for target species that are soluble in water, have high surface adhesion, or vary in observed concentration for any other biochemical reason throughout an exhalation. The target species can be any species that is of diagnostic interest, including but not limited to: alkanes (methane, ethane, etc.), alkenes (ethene, propene, etc.), alcohols (methanol, ethanol, etc.), ketones (acetone, butanone, etc.), aldehydes (formaldehyde, ethanal, etc.), aromatics (benzene, toluene, etc.), dienes (isoprene, etc.), carbon oxides (CO, CO₂), nitrogen compounds (NO, NH₃, etc.), sulfur compounds (H₂S, OCS, etc.), and isotopic versions of the above.

The reference species is not of primary diagnostic interest. Instead good reference species candidates are those that appear in the breath in stable concentrations that differ significantly from the concentration in the environment. Suitable reference species include but are not limited to: water, oxygen and carbon dioxide.

Two variants of the technology have been developed. The first is active sampling via directed oral exhalation, and the second is passive sampling from nasal breath. In the oral variant, the patient places their lips around a mouthpiece (typically a simple tube) and provides a directed, forceful expiration of the tidal and expiratory reserve volume of the lungs. The bulk of the exhaled oral breath is directed through a bypass flow path while a portion of the breath is continuously sampled for spectroscopic analysis. This apparatus can include a patient interface providing biofeedback relating to oral exhalation sample quality. In the nasal variant, the patient wears a nasal cannula that provides continuous suction for sample extraction from the breath jets exiting the nostrils during tidal breathing.

FIG. 2 shows an exemplary embodiment for oral breath sampling. Here the patient provides a breath sample via mouthpiece 142. The breath sample passes through heated tube 140 and gas flow is split between a bypass path including flow meter 134 and a sample path including filter 130. In the sample path, gas flow through optical cell 126 is controlled by pump 110 and valves 112 and 128. Gas flow in the sample path is measured by flow meter 114 and pressure transducer 116. A laser source 118 provides light to optical cell 126, and optical absorption in the cell is measured with optical detector 120. Optionally, mirrors 122 and 124 may be present if/as needed to direct optical beams entering and/or exiting optical cell 126. The above described components are disposed within a temperature controlled environment 132. Computer 136 and biofeedback display 138 do not need to be within temperature controlled environment 132.

In an experimental example of this approach, ammonia was the target species and carbon dioxide was the reference species. Coherent laser light was directed through a multi-pass optical cell (L_{eff}=76 m) and the wavelength-dependent absorption of the incident light intensity was related to molecular abundance of an absorbing gas species through the Beer-Lambert relation. A quantum cascade laser, centered near 10.34 μm was scanned by injection current modulation which yields a simultaneous scan of output intensity and wavelength, enabling access to discrete rovibrational transitions of NH₃ and CO₂. The absorption transitions were each fit with a Voigt line shape function which yields an integrated area (Aᵢ) that can be related directly to concentration or species mole fraction by x_{abs}=Aᵢ/SᵢPL, where Sᵢ is the tabulated line strength of a given transition i, P is the gas pressure, and L is the path length. The laser was scanned at a rate of 1 kHz, yielding effectively instantaneous detection of both species in the same gas volume at a rate much faster than the time scales of exhalation. Post-processing and real-time display in this experiment occurred at a rate of approximately 10 Hz. Ammonia concentration in exhaled breath was measured with a sensitivity of 5 parts per billion with this time-resolved and continuous approach.

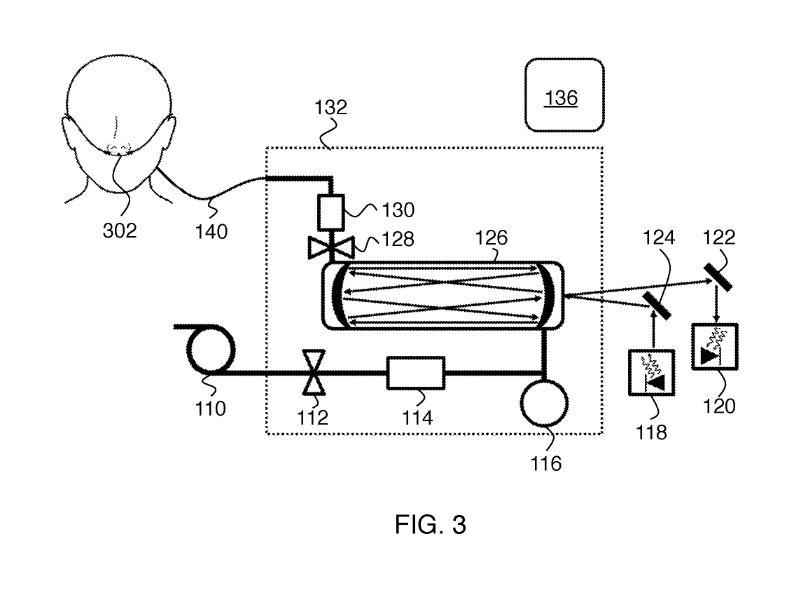

FIG. 3 shows an exemplary embodiment for nasal breath sampling. This example is similar to the example of FIG. 2, except for the following differences. Instead of mouthpiece 142 on FIG. 2, a nosepiece 302 is employed on FIG. 3. A bypass gas flow path as on FIG. 2 is not needed for nasal breath sampling. Similarly, a biofeedback display as on FIG. 2 is not needed for nasal breath sampling.

The sampling system of both variants are designed to preserve the phase-dependent molecular concentrations within an oral/nasal breath sample. The mouth-/nosepiece and all flow paths within both variants are preferably temperature controlled and constructed of inert materials to minimize the surface adsorption of relevant molecular species and eliminate the condensation of water vapor. Each flow path is preferably instrumented with a flow meter to monitor variations in flow rate during exhalation and aid in the identification of the breath phase.

The sampled breath is passed through an optical cell for time-resolved spectroscopic analysis. The temperature, pressure, and flow conditions within the cell can be controlled through heaters, a vacuum pump, valves, thermocouples, flow meters, a pressure transducer, and PID (proportional-integral-derivative) controllers. The pressure and temperature set points are preferably chosen to optimize the absorption spectra of the target and reference breath species. In order to resolve the intra-breath composition, the measurement bandwidth must exceed the relevant breath time scales. To this end, the flow rate and cell volume are chosen to achieve a shorter optical cell flow-through time than relevant breath time scales (<1 sec). Spectroscopic measurement of the breath composition is performed effectively instantaneously at approximately 1 kHz, while post-processing and real-time display occur at approximately 10 Hz. This effectively provides an overall time constant for the concentration measurements of 1 second or less (with most of that being from gas handling).

The spectroscopic measurement of the concentration of target and reference species in the sampled gas is achieved using laser absorption spectroscopy (LAS). Coherent light from one or more lasers is passed through a multi-pass optical cell and the wavelength-dependent absorption of the incident light intensity is related to molecular abundance of an absorbing gas species through the Beer-Lambert relation. The wavelength of the laser output may be tuned to probe discrete transitions within the absorption spectra of one or more species of interest. One or more transitions is selected for each gas species of interest based upon sensitivity, dynamic range, interference, and wavelength accessibility considerations. Tuning techniques including scanned direct absorption spectroscopy and wavelength modulation spectroscopy can be employed to improve the bandwidth, sensitivity, selectivity, and noise-rejection characteristics of the measurement. Alternatively, other laser absorption spectroscopy techniques such as photoacoustic spectroscopy (PAS) or cavity enhanced absorption spectroscopy (CEAS) may also be employed for measurement of the composition of the sampled breath.

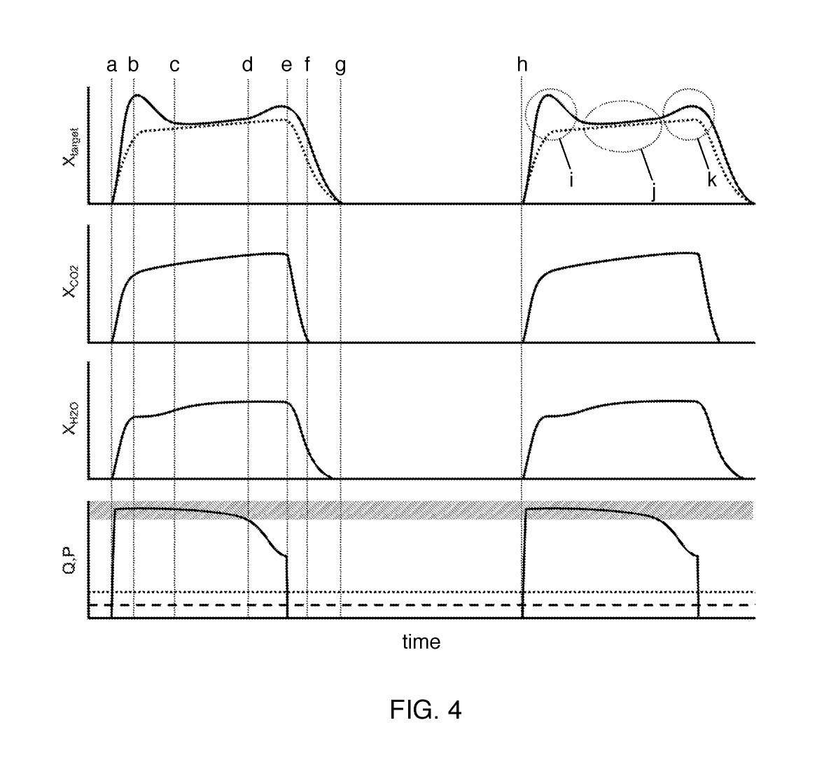

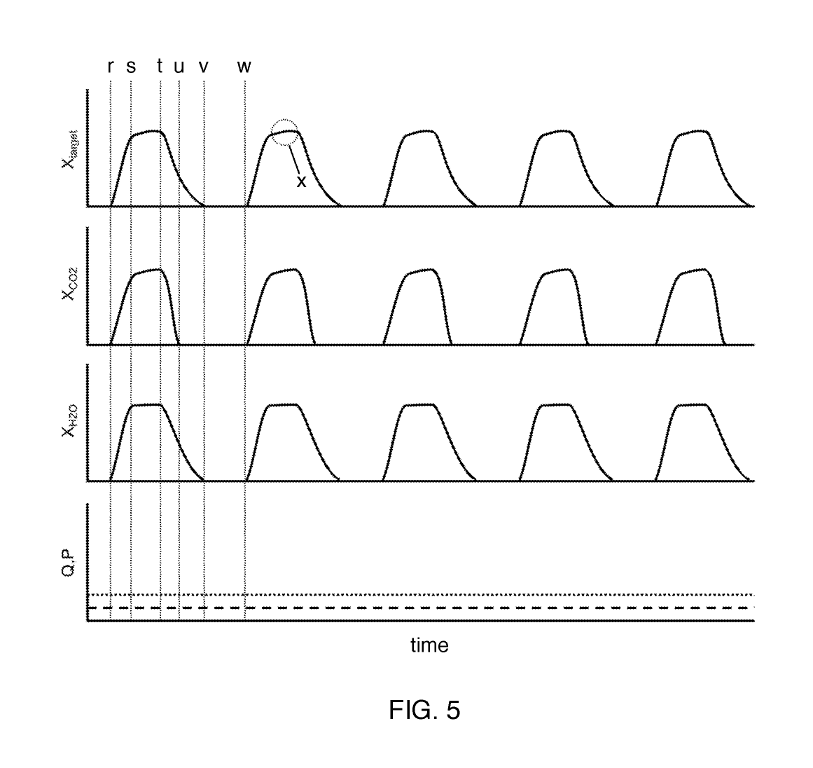

Illustrative time-resolved results from direct oral sampling and passive nasal sampling are presented in FIGS. 4 and 5, respectively. Here X_{target} is target concentration, X_{CO2} is carbon dioxide concentration, X_{H2O} is water concentration, Q is gas flow rate and P is gas pressure.

On FIG. 4, the dashed line on the Q,P plot is sample pressure and the dotted line is sample flow rate (both measured at 114 on FIG. 2). The solid line on the Q,P plot is bypass flow rate (measured at 134 on FIG. 2). The shaded region on the Q,P plot is the optimal range for bypass flow rate. The solid line on the X_{target} plot relates to oral cavity production and the dashed line on the X_{target} plot relates to no oral cavity production.

More specifically, the dashed line represents a case where the concentration of the target species in the breath is derived solely from gas exchange between the alveoli and the blood. The solid line represents a case where the concentration of the target species in the breath is derived from both blood-alveoli gas exchange and generation within the oral cavity from an independent source (e.g., bacteria, food/drink remnants, mouth pH, etc.). Oral cavity production is limited to a subset of possible target species and may change over time and vary between individuals. The relative contribution of the oral cavity gas to the target species concentration varies throughout the exhalation. Intra-breath analysis provides a real-time mechanism to distinguish the respective sources of the target species.

Here the start of a first breath is referenced as ‘a’, the time when the optical cell is flushed is referenced as ‘b’, the end of the oral cavity gas is referenced as ‘c’, and a drop in breath flow rate is referenced as ‘d’. The end of the first breath is referenced as ‘e’, the time when the optical cell is flushed (of the first breath sample) is referenced as ‘f’, the time when adsorbed molecules are eliminated is referenced as ‘g’, and the time a second breath starts is referenced as ‘h’. Time span ‘i’ relates to excess oral cavity contribution in the early part of a breath. Time span ‘k’ relates to excess oral cavity contribution in the late part of a breath. Time span ‘j’ is the gated part of the sample (starting at ‘c’ and ending at ‘d’), deemed to be most relevant.

On FIG. 5, the dashed line on the Q,P plot is sample pressure and the dotted line is sample flow rate (both measured at 114 on FIG. 3). Here the start of a first breath is referenced as ‘r’, the time when the optical cell is flushed is referenced as ‘s’, and the end of the first breath is referenced as ‘t’. The time when the optical cell is flushed (of the first breath sample) is referenced as ‘u’, the time when adsorbed molecules are eliminated is referenced as ‘v’, and the time a second breath starts is referenced as ‘w’. Time span ‘x’ is the gated part of the sample (starting at ‘s’ and ending at ‘t’), deemed to be most relevant.

Processor 136 on FIGS. 2 and 3 is preferably configured to automatically select a relevant part of the concentration measurements of the at least one target chemical species according to one or more gating criteria including but not limited to: concentration measurements of reference chemical species, concentration measurements of target chemical species, gas flow rate in the breath sample collection unit and gas pressure in the breath sample collection unit. Thus the processor is configured to automatically select relevant parts of the continuous data, such as regions ‘j’ and ‘x’ on FIGS. 4 and 5 respectively. As described in greater detail below, this selection of relevant parts of the data can include, as a special case, elimination of samples deemed to have inadequate sampling quality.

This technology has five key aspects: 1) Measurement of phase-dependent molecular concentrations over an entire oral/nasal exhalation; 2) Correction for breath sample contamination by environmental air; 3) Real-time patient biofeedback for improved sample quality during directed oral sampling; 4) Single breath quality assessment and rejection and 5) Adaptive gating of single-breath concentration time histories for improved correlation to systemic concentrations of target species. These aspects are discussed in more detail below. Most details of these aspects apply equally to the oral and nasal approaches. Details that only apply to one or the other are indicated accordingly.

### 1) Measurement of Phase-Dependent Molecular Concentrations Over an Entire an Oral/Nasal Exhalation

The concentrations of molecular species within the breath vary depending upon the depth and phase of the exhalation. As a patient exhales, the expelled breath is sourced from multiple progressively deeper volumes (e.g., oral/nasal cavity, anatomical dead space, alveolar sacs) where concentrations may vary widely and convolute the correlation to systemic levels. A sampling system that preserves the relative phase of the respective exhaled volumes in combination with a measurement technique with sufficient bandwidth may be used to resolve the phase-dependent molecular concentrations and their respective rates of change over the duration of an entire single exhalation. The preserved-phase history of species concentrations coupled with simultaneous flow rate measurements permits the identification and analysis of systemically relevant breath phases, the correction for sample contamination, the incorporation of biofeedback to improve sample quality, and the rejection of poor quality samples.

A first feature of this aspect is preservation of phase-dependent molecular concentrations within an oral/nasal breath sample. Several design considerations contribute to achieving this goal. Longitudinal mixing of gas flow from patient to optical cell is minimized by use of smooth flow paths, minimized dead-volume, minimized recirculation, and suction-induced flow. The flow-through time of the optical cell is preferably shorter than relevant breath time scales to eliminate smoothing of variations in molecular concentrations due to stratification within measurement volume. System flow-through time is preferably shorter than diffusion time scales. For oral sampling, the breath sampling system preferably includes a sampling flow path that extracts gas at a constant flow rate from a bypass flow path (as in the example of FIG. 2). For nasal sampling, a preferred sampling approach is suction-based sample collection from breath jet near nasal/oral orifice to minimize sample mixing.

A second feature of this aspect is preservation of relative phase of multiple molecular species concentrations. There is preferably a common measurement volume (optical cell) and preceding flow path for all gas species, and the analysis instrument should be capable of providing simultaneous multi-species concentration measurements.

A third feature of this aspect is sufficient measurement bandwidth. Concentration measurement time is preferably less than flow-through time of the measurement volume (optical cell).

A fourth feature of this aspect is accounting for return of measured concentrations to environmental levels during the interval between exhalations. Molecular adsorption sites are preferably minimized through use of inert tubing materials of minimum length and heated above the dew point of breath. The volumetric flow rate through measurement volume (optical cell) is preferably maximized to permit multiple flushes of measurement volume with environmental air during interval between breaths. For oral breath measurement with directed sample collection, subsequent breath samples may be delayed until measured concentrations return to environmental levels.

A fifth feature of this aspect is maintenance of constant sample gas conditions within the optical cell during the patient's oral/nasal breath cycle. This can be accomplished by use of pressure and flow rate regulation through valves upstream and downstream from the optical cell. Temperature regulation through temperature-controlled flow paths and the optical cell can also be employed. For oral breath measurement with directed sample collection, side-stream sample extraction from bypass breath flow path can be used to mitigate breath induced pressure/flow rate fluctuations.

### 2) Correction for Breath Sample Contamination by Environmental Air

Sampling of the breath is susceptible to contamination by environmental air and this contamination is preferably corrected for to ensure measurements are reflective of systemic concentrations of the target species. In the case of directed oral sampling, the contamination is minimal due to the sealed interface between the patient's lips and the mouthpiece. However, in the case of passive nasal sampling the contamination can form a substantial portion of the breath sample depending upon how well positioned the holes in the nose piece are in relation to the breath jet near the nostrils. The processor can be configured to automatically correct for contamination of breath samples by environmental air according to at least the concentration measurements of the reference chemical species. To appropriately correct for contamination by environmental air, two approaches may be employed.

Approach 1: Sample Contamination Correction and Normalization Using Reference Species with Unknown but Stable Concentration in Breath (e.g., CO₂ or H₂O)

The measured concentrations of the target species, χₛ, and the reference species, χᵣ, may be expressed as the sum of their respective concentrations in the breath, χ_{xb}, and in the environment, χₓₑ, weighted by the fractional contamination a of the breath gas by the environment gas:

χₛ=aχ_{sb}+(1−a)χₛₑ,

and

χᵣ=aχ_{rb}+(1−a)χᵣₑ.

These equations may be combined to express the concentration of the target species within the breath as

$${χ}_{sb} = ( {χ}_{s} - {χ}_{se} ) ( \frac{{χ}_{rb} - {χ}_{re}}{{χ}_{r} - {χ}_{re}} ) + {χ}_{se} . \qquad ( 1 )$$

The environmental concentrations of the target species, χₛₑ, and the reference species, χᵣₑ, may be measured independently prior to acquiring a breath sample.

In order to eliminate the dependence on the unknown breath concentration of the reference species, the concentration of the target species within the breath may be normalized by the breath concentration of the reference species as

$$\frac{{χ}_{sb}}{{χ}_{rb}} = ( \frac{{χ}_{s} - {χ}_{se}}{{χ}_{rb}} ) ( \frac{{χ}_{rb} - {χ}_{re}}{{χ}_{r} - {χ}_{re}} ) + \frac{{χ}_{se}}{{χ}_{rb}} .$$

Given the selection of target and reference species with breath concentrations much larger than the environmental concentrations (χ_{rb}>>χᵣₑ and χ_{sb}>>χₛₑ) the reference species normalized concentration of the target species in the breath may be simplified as

$$\frac{{χ}_{sb}}{{χ}_{rb}} = \frac{{χ}_{s} - {χ}_{se}}{{χ}_{r} - {χ}_{re}} .$$

CO₂ is a suitable reference species candidate for correction and normalization due to significantly elevated CO₂ concentrations in the breath (typically 4-5%) with respect to the environmental concentration (approximately 400 ppm). Normalization by CO₂, as described above, is necessary as the exact CO₂ concentration in the breath of the patient is unknown and may vary somewhat over long time scales. The CO₂ concentration is dependent upon factors including posture, activity, blood-pH, and various disease states.

H₂O is also a suitable reference species candidate for correction and normalization due to elevated H₂O concentration in the breath (approximately 5%) with respect to the environmental concentration in a climate controlled room (approximately 0.5%). The concentration of water in the tidal breath of the patient is determined by the saturated vapor pressure of water at the temperature of the breath as it exits the mouth or nose. This simple relationship for exhaled water concentration allows for an additional correction approach given below.

Approach 2: Sample Contamination Correction Using Reference Species with Known and Stable Concentration in Breath (e.g., H₂O)

Due to interaction of the breath with the moist mucosal layers of the lungs, trachea, oral cavity, and nasal cavity, the breath reaches a saturated H₂O concentration dictated by the temperature of the breath as it exits the mouth or nose,

$${χ}_{rb} = \frac{{P}_{sat} ( {T}_{b} )}{{P}_{atm}} .$$

By measuring the temperature of the patient's breath as it exits the mouth or nose, the concentration of H₂O in the breath of the patient may be determined from thermodynamic tables. With a known concentration of the reference species, Equation 1 may be used to calculate the corrected breath concentration of the target species, χ_{sb}.

Suitable reference species have stable but disparate breath and environment concentrations. Concentration of reference species is preferably stable within the measurement environment (e.g., room) during the period of device use. Concentration of reference species is also preferably stable within the breath during the period of device use. The concentration of the reference species in the breath is preferably much larger than the concentration of the reference species in the environment. The difference in the concentration of the reference species in the breath vs. the environment is preferably much larger than the resolution of the measurement of the reference species. The concentration of the target species in the breath is preferably much larger than the concentration of the target species in the environment. The difference in the concentration of the target species in the breath vs. the environment is preferably much larger than the resolution of the measurement of the target species.

These correction methods both make use of measurement of phase-dependent molecular concentrations over all or part of an entire oral/nasal exhalation where the reference species is one of the measured concentrations, as described above, combined with measurement or knowledge of target and reference species concentrations in the environment. For Approach 2, measurement of exhaled breath temperature immediately following exit from nose/mouth is performed.

### 3) Real-Time Patient Biofeedback for Improved Sample Quality During Directed Oral Sampling

During an oral breath measurement, the ideal patient would provide a forced expiration of the complete tidal volume and expiratory reserve volume at a stable flow rate several times greater than the sampling flow rate but not so high as to impact the stability of the measurement volume pressure. A consistent breath profile minimizes breath-to-breath and patient-to-patient measurement variability. In practice, oral breath samples provided by patients may vary significantly in duration, flow rate, continuity, and phase of respiratory cycle. Pre-collection breath coaching may reduce breath-to-breath variability and improve the patient's ability to provide an optimal oral breath sample; however, active feedback during the exhalation provides the most significant improvements.

A real-time biofeedback display provides the patient with active guidance to correct their exhalation as it proceeds. The information employed in the display may include the exhaled breath flow rate time-history, instantaneous flow rate, breath duration, and/or cumulative exhaled volume. Additionally, the display may include target values, thresholds, or windows for each of these parameters with a visual indicator guiding the patient toward the characteristics of an optimal breath exhalation. These values may be presented directly or in an abstracted easy-to-understand visual form such as graphs, bar plots, changing colors, animations, etc.

Features of preferred embodiments include the following, either individually or in any combination: A breath sampling system where a sampling flow path extracts gas at a constant flow rate from a bypass flow path; A fast response flow rate measurement on both the bypass and sampling flow paths; A real-time display presenting the patient with the target exhaled breath flow rate, the present exhaled breath flow rate, and guidance to adapt the exhalation flow rate to meet the target; and/or A real-time display presenting the patient with the target exhaled breath duration, the present exhaled breath duration, and guidance to adapt the exhaled breath duration to meet the target.

### 4) Single Breath Quality Assessment and Rejection

The preservation and measurement of the phase-dependent molecular concentrations within an oral/nasal breath sample permit the quality of the breath sample to be assessed and potentially rejected to ensure accurate measurements of concentration. Additionally, the performance of the device may be actively assessed to ensure suitable device performance. Quality assessment and rejection of samples may be based upon the criteria listed below, either individually or in any combination:

i) Magnitude, stability, and continuity of flow;
ii) Target and reference species concentrations and stability;
iii) Duration of exhalation;
iv) Presence or absence of distinct breath phases (e.g., oral cavity air, tidal breath, expiratory reserve volume);
v) Duration of distinct breath phases (e.g., oral cavity air, tidal breath, expiratory reserve volume);
vi) Fraction of sample dilution by environmental air;
vii) Return of measured concentrations to environmental levels between breath samples;
viii) Rate of decay of measured concentrations to environmental levels between breath samples; and
ix) Sample volume temperature, pressure, and/or flow rate.

Quality assessment and rejection of samples may occur in real-time or as a part of a post-processing procedure. Sample quality assessment makes use of measurement of phase-dependent molecular concentrations (target and possibly reference species) over all or part of an entire oral/nasal exhalation as described above. Measurements of the temperature, pressure, and flow rate within the measurement volume with sufficient response to resolve transients in exhaled breath can also be employed. For oral breath measurement with directed sample collection, measurement of exhaled breath flow rate with sufficient response to resolve transients in exhaled breath can be employed.

### 5) Adaptive Gating of Single-Breath Concentration Time Histories for Improved Correlation to Systemic Concentrations of Target Species

Time-resolved single-breath analysis provides the capability to observe, identify, and quantify the phases of the exhaled breath through the simultaneous measurement of target species, reference species, and breath flow rates. This capability permits individual exhaled breaths with unique flow rate, duration, composition, and phases to be adaptively gated for the selection of relevant portions of the breath sample. For example, the exhalation may be gated to select portions of the breath with maximum contribution from alveolar air and thus greatest utility as an indicator of the systemic concentrations of the target species. Detailed considerations for the oral sampling and nasal sampling cases are given below.

As illustrated in FIG. 4, the target species concentration during a typical oral breath sample will typically progress through several characteristic phases. Initially, the target concentration will rise rapidly (‘a’ to ‘b’ on FIG. 4) as the environmental air in the measurement volume is replaced by the early portion of the exhaled breath. The initially exhaled portion of the breath is primarily a combination of oral cavity air and anatomical dead space air (tidal air from the previous inhalation with little contribution from alveolar air). Due to the lack of alveolar air in this breath phase, the concentration of the target species of systemic origin and CO₂ are lower. H₂O concentration during this phase is also reduced due to the limited time for anatomical dead space tidal air to reach saturation. During this early breath phase the concentration of the target species may be reduced if the only source of the target species is alveolar air; however, some species (e.g., ammonia) may also be orally generated and thus due to the large fraction of oral cavity air during the early breath phase, the concentration of the target species may be significantly elevated.

Once the contribution from the oral cavity has been cleared (i.e., after ‘c’ on FIG. 4), the concentrations of the target species, CO₂, and H₂O will rise as the fractional contribution from alveolar air increases. This corresponds to a transition from the tidal volume of the lungs to the expiratory reserve volume. The H₂O concentration will increase until it reaches the saturation vapor pressure dictated by the temperature of the breath at the exit of the mouth.

The exhaled breath is composed of a larger fraction of alveolar air as the patient reaches the end of their expiratory reserve volume; however, at this point in the exhalation the patient's ability to provide a steady flow rate is diminished and either the patient will terminate the breath or drop below the required breath flow rate (e.g., ‘d’ on FIG. 4). The reduced flow rate may lead to an increase in the concentration of the target species, if the rate of oral production of the target species is significant relative to the exhaled flow rate. Once the exhalation is completed, the measured concentrations of all species drop rapidly as the measurement volume is replaced with environmental air.

Given the ability to identify these respective phases of a directed oral exhalation, the time history of the target species may be gated to isolate the portion of the breath with the maximum contribution from the alveolar air and minimum contribution from the oral cavity. In the example of FIG. 4, this gated region starts at ‘c’ and ends at ‘d’, and is also schematically referenced as ‘j’ in the second breath. Given a suitable exhalation, this region may be gated based upon the following criteria, either individually or in any combination:

i) Period of bypass flow rate within optimal flow rate range or some fraction thereof;
ii) Period between early and late oral contributions;
iii) Period where target species concentration exceeds some relative threshold;
iv) Period where reference species concentration exceeds some absolute or relative threshold.
v) Period where the rate of change of the target species concentration exceeds some relative threshold; and
vi) Period where the rate of change of the reference species concentration exceeds some absolute or relative threshold.

The concentration of the target species within the gated region may then be processed to produce a single value to represent the systemic concentration for a single breath or a series of breaths.

The characteristic phases for passively collected nasal breath vary considerably from those of a directed oral breath. As illustrated in FIG. 5, the passively collected nasal breaths are shorter and are typically composed only of tidal breath with a lower fraction of alveolar air. Additionally, due to the sampling from the jet near the nostrils a greater fraction of environmental air is expected in the sample and thus the concentration of the target species in the sample is reduced.

Initially, the target species concentration rises rapidly (‘r’ to ‘s’ on FIG. 5) as the environmental air in the measurement volume is replaced by the early portion of the exhaled breath. The initially exhaled portion of the breath is primarily anatomical dead space tidal air from the previous inhalation with little contribution from alveolar air. Due to the lack of alveolar air in this breath phase, the concentration of the target species of systemic origin and CO₂ are lower. Typically, there is no significant contribution to the target species concentration from the nasal cavity.

As the exhalation progresses (‘s’ to ‘t’ on FIG. 5), the concentrations of the target species, CO₂, and H₂O will rise more slowly as the fractional contribution from alveolar air increases. Due to the exhalation of only tidal breath, the rise in concentration is limited by a reduced contribution from the expiratory reserve volume. The H₂O concentration will increase until it reaches the saturation vapor pressure dictated by the temperature of the breath at the exit of the nostrils. In comparison to oral breath, the H₂O concentration in nasal breath saturates more rapidly due to increased interaction with moist mucosal tissue but reaches a lower saturation concentration due to the lower temperature of the exhaled nasal breath.

At the end of the tidal exhalation (after ‘t’ on FIG. 5), the measured concentrations of all species drop rapidly as the measurement volume is replaced with environmental air.

Given the ability to identify these respective phases of a passive nasal exhalation, the time history of the target species may be gated to isolate the portion of the breath with the maximum contribution from the alveolar air. Given a suitable exhalation, this region may be gated based upon the following criteria, either individually or in any combination:

i) Period where target species concentration exceeds some relative threshold;
ii) Period where reference species concentration exceeds some absolute or relative threshold.
iii) Period where the rate of change of the target species concentration exceeds some relative threshold; and
iv) Period where the rate of change of the reference species concentration exceeds some absolute or relative threshold.

The concentration of the target species within the gated region may then be processed to produce a single value to represent the systemic concentration for a single breath or a series of breaths.

Adaptive gating makes use of measurement of phase-dependent molecular concentrations (target and possibly reference species) over all or part of an entire oral/nasal exhalation as described above. For oral breath measurement with directed sample collection, measurement of exhaled breath flow rate with sufficient response to resolve transients in exhaled breath can be employed.

1. Apparatus for breath sampling, the apparatus comprising:
a breath sample collection unit;
an optical spectrometer configured to receive gas from the breath sample collection unit and to provide time-resolved and continuous intra-breath concentration measurements of at least one target chemical species.

2. The apparatus of claim 1, wherein the optical spectrometer is further configured to provide time-resolved and continuous intra-breath concentration measurements of at least one reference chemical species, and wherein the measurements of the target species and the reference species are synchronized.

3. The apparatus of claim 2, wherein the reference chemical species is selected from the group consisting of: water, oxygen and carbon dioxide.

4. The apparatus of claim 2, further comprising a processor configured to automatically select a relevant part of the concentration measurements of the at least one target chemical species according to one or more gating criteria selected from the group consisting of: concentration measurements of the at least one reference chemical species, concentration measurements of the at least one target chemical species, gas flow rate in the breath sample collection unit and gas pressure in the breath sample collection unit.

5. The apparatus of claim 4, wherein the processor is configured to automatically correct for contamination of breath samples by environmental air according to at least the concentration measurements of the at least one reference chemical species.

6. The apparatus of claim 1, wherein the breath sample collection unit is configured for active sampling via oral exhalation.

7. The apparatus of claim 6, further comprising a patient interface providing biofeedback relating to oral exhalation sample quality.

8. The apparatus of claim 1, wherein the breath sample collection unit is configured for passive sampling from nasal breath.

9. The apparatus of claim 1, wherein the target chemical species is selected from the group consisting of: ammonia, acetone, formaldehyde, ethanol, methane, alkanes, alcohols, aldehydes, dienes and ketones.

10. The apparatus of claim 1, wherein a time constant for the concentration measurements is 1 second or less.
